(19) United States
(12) Patent Application Publication
(10) Pub. No.: US 2026/0115492 A1
(43) Pub. Date: Apr. 30, 2026

(54) RADIOTHERAPY DELIVERY DEVICE AND METHODS OF PERFORMING RADIOTHERAPY

(71) Applicant: BARD PERIPHERAL VASCULAR, INC., Franklin Lakes, NJ (US)

(72) Inventors: Matthew Cartwright, Chandler, AZ (US); Summer Ford, Queen Creek, AZ (US); Genevieve Messina, Mesa, AZ (US); William E. Parmentier, Gilbert, AZ (US); Kyra Temple, Mesa, AZ (US); Peng Zheng, Chandler, AZ (US)

(73) Assignee: BARD PERIPHERAL VASCULAR, INC., Franklin Lakes, NJ (US)

(21) Appl. No.: 19/473,923

(22) PCT Filed: Apr. 10, 2023
(86) PCT No.: PCT/US2023/018019

§ 371 Date: Oct. 9, 2025

(51) Int. Cl. A61N 5/10

(52) U.S. Cl. CPC A61N 5/1007; A61N 2005/1021

(57) ABSTRACT

A radiotherapy delivery device includes a body having a first end and a second end and a catheter having a lumen extending between a proximal end and a distal end. The proximal end of the catheter is coupled to the second end of the body and the distal end of the catheter includes a cutting tip that penetrates a target site. A sealed pouch containing radioactive particles is positioned within the lumen of the catheter, and a puncture shaft is positioned coaxially within the body. The puncture shaft advances in a longitudinal direction into and through the sealed pouch to release the radioactive particles. A syringe is fluidly coupled to the lumen of the catheter, and the radioactive particles released by the sealed pouch are deposited into the target site when fluid is supplied from the syringe and into the lumen of the catheter.

### TECHNICAL FIELD

The present disclosure relates to radiotherapy devices, and, more particularly, to radiotherapy delivery devices and methods of delivering radiotherapy.

### BACKGROUND

Radiotherapy involves placing a small amount of radioactive material inside the body, such as near cancer cells or within a tumor. Unlike external radiation treatment such as electron beam irradiation, radiotherapy enables delivery of radiation to a small, targeted area for treatment of cancerous tissue while minimizing damage to surrounding healthy tissue. Delivering radioactive particles directly into tumor tissue further reduces other adverse effects on healthy tissue. However, many radiotherapy delivery devices are associated with complications, such as pneumothorax, and are incapable of providing radiotherapy to a plurality of treatment locations during the same procedure. Furthermore, these radiotherapy delivery devices often utilize biodegradable mesh markers, which can be difficult to accurately position within a target site.

### SUMMARY

An object of the present disclosure is to provide a radiotherapy device that can minimize the risk of complications and allow for accurate placement of radioactive particles and/or microspheres in multiple treatment locations during the same procedure.

In one embodiment, a radiotherapy delivery device is disclosed. The delivery device may include a body having a first end and a second end, and a catheter having a lumen extending between a proximal end and a distal end. The proximal end of the lumen may be coupled to the second end of the body and the distal end may include a cutting tip configured to penetrate a target site. The radiotherapy delivery device may further include a sealed pouch containing radioactive particles positioned within the lumen of the catheter, and a puncture shaft positioned coaxially within the body. The puncture shaft may be configured to advance in a longitudinal direction from the first end of the body towards the second end of the body such that the puncture shaft advances into and through the sealed pouch and releases the radioactive particles. The radiotherapy delivery device may also include a syringe fluidly coupled to the lumen of the catheter, such that the radioactive particles released by the sealed pouch are deposited into the target site when fluid is supplied from the syringe and into the lumen of the catheter.

In another embodiment, a radiotherapy delivery device is disclosed. The delivery device may include a body having a first end and a second end, and a catheter having a lumen extending between a proximal end and a distal end. The proximal end of the lumen may be coupled to the second end of the body and the distal end may include a cutting tip configured to penetrate a target site. The radiotherapy delivery device may further include a sealed pouch containing radioactive particles positioned within the lumen of the catheter, and a piston shaft positioned coaxially within the body. The piston shaft may include a piston and may be configured to translate in a longitudinal direction between an engaged position in which the piston contacts the sealed pouch and a disengaged position in which the piston is withdrawn from the sealed pouch. The radiotherapy delivery device may further include a puncture shaft positioned coaxially within the piston shaft, with the puncture shaft being configured to advance in the longitudinal direction from the first end of the body towards the second end of the body such that the puncture shaft advances into and through the sealed pouch and releases the radioactive particles. In the engaged position, advancement of the piston shaft in the longitudinal direction may force radioactive particles from the sealed pouch and towards the target site.

In yet another embodiment, a method of radiotherapy delivery is disclosed. The method may involve depositing a sealed pouch containing radioactive particles within a radiotherapy delivery device. The radiotherapy delivery device may include a body having a first end and a second end and a catheter having a lumen extending between a proximal end and a distal end, wherein the proximal end of the catheter is coupled to the second end of the body. The method may further involve the steps of inserting the catheter into a target site and advancing a puncture shaft coupled to the first end of the body into and through the sealed pouch. Once the pouch is punctured, the method may further involve releasing the radioactive particles in the sealed pouch and supplying a fluid to the catheter lumen with a syringe such that the radioactive particles are moved through the catheter lumen and into the target site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a perspective view of an illustrative radiotherapy delivery device, according to one or more embodiments shown and described herein;

FIG. 2A depicts a cross-sectional front view of the radiotherapy delivery device of FIG. 1 in a first position, according to one or more embodiments shown and described herein;

FIG. 2B depicts a cross-sectional front view of the radiotherapy delivery device of FIG. 1 in a second position, according to one or more embodiments shown and described herein;

FIG. 2C depicts a cross-sectional front view of the radiotherapy delivery device of FIG. 1 in a third position, according to one or more embodiments shown and described herein;

FIG. 3A depicts a cross-sectional front view of another embodiment of a radiotherapy delivery device in a first position, according to one or more embodiments shown and described herein;

FIG. 3B depicts a cross-sectional front view of the radiotherapy delivery device of FIG. 3A in a second position, according to one or more embodiments shown and described herein;

FIG. 4A depicts a cross-sectional front view of another embodiment of a radiotherapy delivery device in a first position, according to one or more embodiments shown and described herein;

FIG. 4B depicts a cross-sectional front view of the radiotherapy delivery device of FIG. 4A in a second position, according to one or more embodiments shown and described herein;

FIG. 4C depicts a cross-sectional front view of the radiotherapy delivery device of FIG. 4A in a third position, according to one or more embodiments shown and described herein;

FIG. 5A depicts another embodiment of a radiotherapy delivery device in a first position, according to one or more embodiments shown and described herein;

FIG. 5B depicts a cross-sectional front view of the radiotherapy delivery device of FIG. 5A in a second position, according to one or more embodiments shown and described herein;

FIG. 5C depicts a cross-sectional front view of the radiotherapy delivery device of FIG. 4A in a third position, according to one or more embodiments shown and described herein; and

FIG. 6 depicts a flow chart of an illustrative method of performing a radiotherapy procedure, according to one or more embodiments shown and described herein;

Reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to radiotherapy delivery devices and methods of performing radiotherapy. The radiotherapy delivery devices described herein may include a body having a first end a second end, and a port for receiving a syringe. The radiotherapy delivery device may further include a catheter, which may be coupled to the second end of the body. A puncture shaft may be coaxially disposed within the body, and may include a puncture handle which may be slidably coupled to a slot formed on an outer surface of the body. The puncture shaft may be translated in a longitudinal direction through the slot, which may in turn advance the puncture shaft through the body of the catheter when the catheter is coupled to the second end of the body. The syringe may include a syringe needle, which may extend through the port and into the lumen of the catheter, such that the syringe and lumen are fluidly coupled.

A pouch containing radioactive particles and/or microspheres may be enclosed within a lumen of the catheter. As the puncture shaft is advanced through the body and catheter, the puncture shaft may contact the pouch, such that the puncture shaft pierces the pouch and releases the radioactive particles and/or microspheres sealed within the pouch. The syringe may be depressed to transfer fluid from the syringe and into the lumen of the catheter. As the fluid flows through the catheter, the advancing fluid may force a volume of radioactive particles and/or microspheres out of the catheter and into a target site.

Embodiments of the present disclosure may be specifically advantageous for providing radiotherapy to a plurality of target sites during a single radiotherapy procedure. By controlling the dosage of radioactive particles and/or microspheres at a target site using fluid from a syringe, a user may easily and accurately deploy a desired volume of radioactive particles and/or microspheres at a particular target site. The radiotherapy delivery device may then be repositioned, and the remaining fluid in the syringe may be used to provide an additional volume of radioactive particles and/or microspheres to a different target site. Furthermore, use of the catheter to position the radiotherapy delivery device further alleviates the need for commonly used biodegradable mesh positioning mechanisms, which can be difficult to accurately place and are incapable of being repositioned to treat multiple target sites in a single procedure.

Embodiments of radiotherapy delivery devices, and methods of radiotherapy procedures will now be described in more detail herein with reference to the drawings and where like numbers refer to like structures.

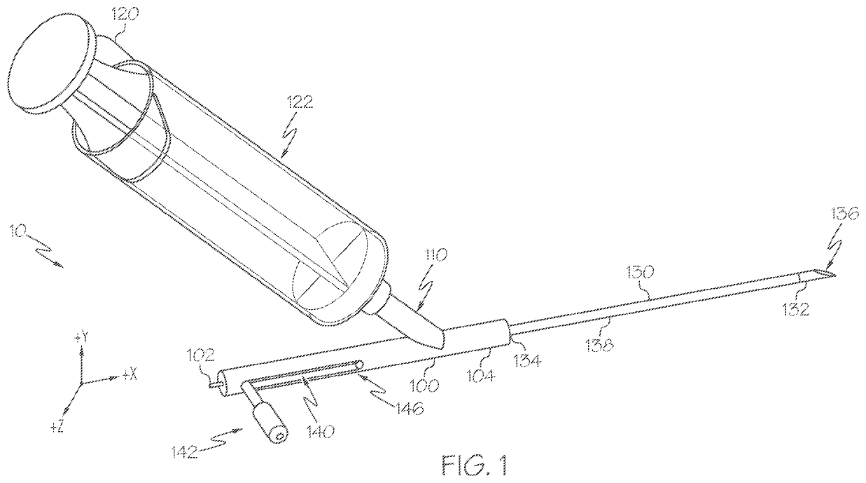

Referring now to FIG. 1, a radiotherapy delivery device 10 is depicted. The radiotherapy delivery device 10 includes a body 100 having a first end 102 and a second end 104. The radiotherapy delivery device 10 further includes a catheter 130, such as a flexible catheter, having a distal end 132, a proximal end 134, and a lumen 138 which extends from the distal end 132 to the proximal end 134. As illustrated in FIG. 1, the proximal end 134 of the catheter 130 may be coupled to the second end 104 of the body 100. In these embodiments, the proximal end 134 of the catheter 130 may be coupled to the second end 104 of the body 100 via any suitable connection (e.g., via threaded connection, adhesive, welding, brazing, etc.).

The body 100 of the radiotherapy delivery device 10 may further include a port 110, which may be shaped, sized, and/or configured to receive a syringe 120. In these embodiments, the syringe 120 may be inserted through the port 110 and into the lumen 138 of the catheter 130 such that the syringe 120 and the catheter 130 are fluidly coupled. By fluidly coupling the syringe 120 to the catheter 130, fluid within the syringe 120 may be dispensed into the lumen 138 of the catheter 130 when the syringe 120 is moved from an extended position to a depressed position, such that, as the syringe is moved from the extended position to the depressed position, fluid is dispelled from the syringe 120 into the catheter 130. In these embodiments, fluid which is dispelled from the syringe 120 and into the lumen 138 of the catheter 130 may flow through the catheter 130 towards the distal end 132 of the catheter 130. The distal end 132 of the catheter 130 may further include a tip, such as a cutting tip 136, which may be used to pierce tissue and position the radiotherapy delivery device 10 at a desired location within a target site. In these embodiments, the syringe 120 and port 110 may be releasably coupled, such that the syringe 120 may be detached from the radiotherapy delivery device 10 and refilled with fluid following the completion of a radiotherapy procedure. For example, the syringe 120 may be threadably coupled to the catheter 130, or coupled via any other similarly releasable mechanism. Additionally, the syringe 120 may be detached during a radiotherapy procedure to refill the syringe 120 with fluid if additional fluid is required to complete the radiotherapy procedure. In the embodiments described herein, the syringe 120 may be a saline syringe, and/or may contain any other fluid suitable for performing radiotherapy procedures.

The radiotherapy delivery device 10 may further include a puncture shaft 140, which may be positioned coaxially within the body 100 of the radiotherapy delivery device 10. In some embodiments, the puncture shaft 140 may have a length Lp which is greater than the length Lb of the body 100, such that the puncture shaft 140 may extend in a longitudinal direction (e.g., in the +/−x indirection of the coordinate axes of FIG. 1) beyond the first end 102 and/or second end 104 of the body 100. In these embodiments, the puncture shaft 140 may extend out of the second end 104 of the body and into the proximal end 134 of the catheter 130 when the catheter 130 is coupled to the body 100, as will be described in more detail herein.

Referring still to FIG. 1, the puncture shaft 140 may include a puncture handle 142, which may be used to translate the puncture shaft 140 in the longitudinal direction (+/-x as shown in the coordinate axis of FIG. 1) through the body 100 and catheter 130 of the radiotherapy delivery device 10. In these embodiments, the puncture handle 142 may be translate within a puncture slot 146 formed in the body 100 of the radiotherapy delivery device 10. The puncture slot 146 may include a distal end 146a and a proximal end 146b, that may define a distance the puncture handle 142 may translate the puncture shaft 140 in the longitudinal direction (+/−x as shown in the coordinate axis of FIG. 1). For example, the puncture shaft 140 may be translatable in the longitudinal direction between a retracted position and an advanced position. In these embodiments, the retracted position may correspond to a position of the puncture shaft 140 when the puncture handle 142 is positioned at or near the distal end 146a of the puncture slot 146 (e.g., moved in the +x direction as shown in the coordinate axis of FIG. 1) The advanced position may correspond to the position of the puncture shaft 140 when the puncture handle 142 is positioned at or near the proximal end 146b of the puncture slot 146 (e.g., moved in the-x direction as shown in the coordinate axis of FIG. 1). The translation of the puncture shaft 140 will be described in more detail herein.

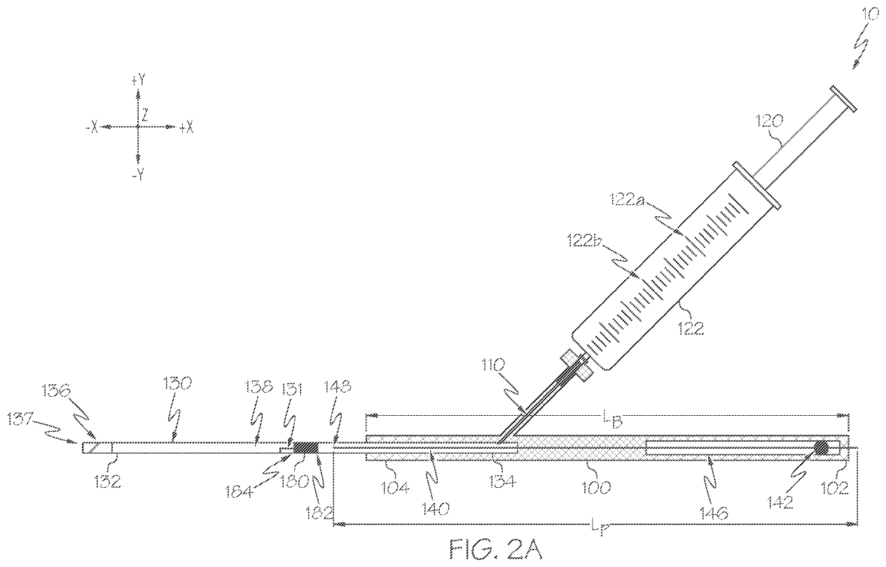

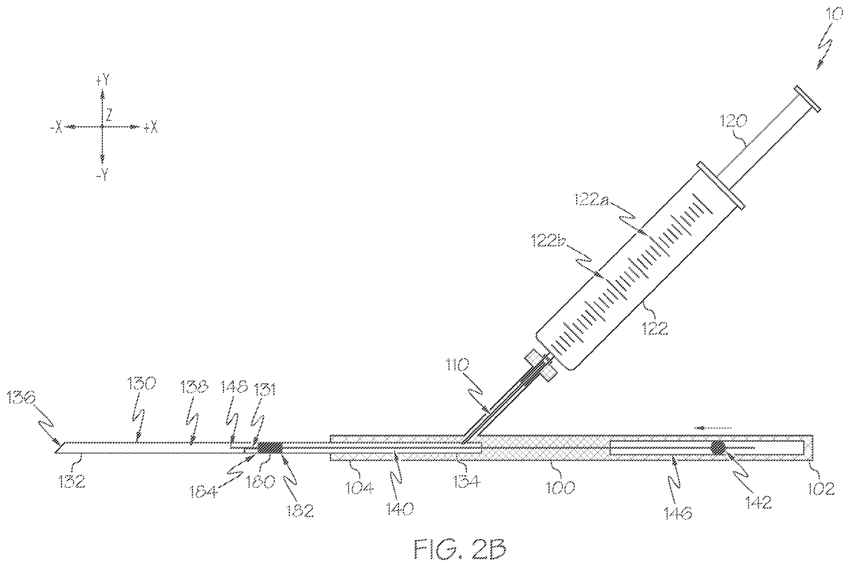

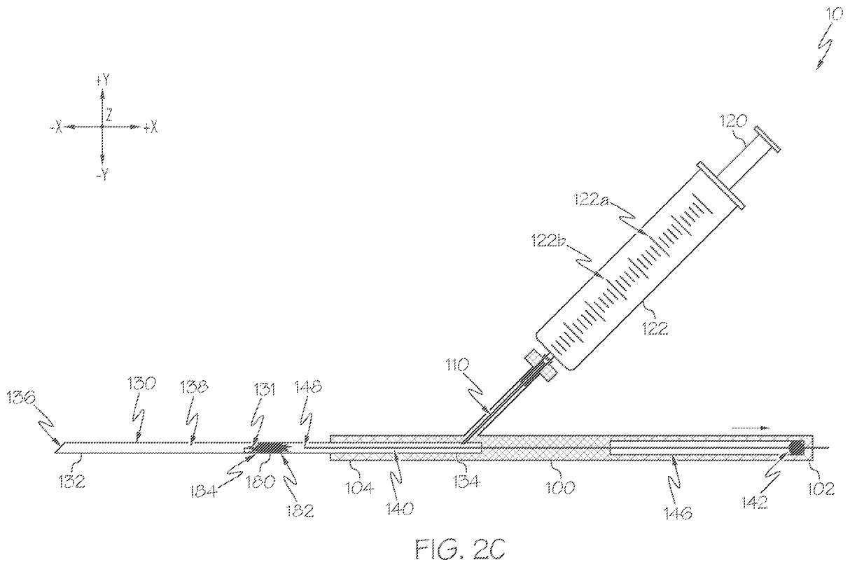

Referring now to FIG. 2A, a cross-sectional front side view of the radiotherapy delivery device 10 of FIG. 1 is depicted. As illustrated in FIG. 2A, the radiotherapy device may further include a pouch 180, such as a sealed pouch, which may contain radioactive particles and/or microspheres. The pouch 180 may be sealed in any manner sufficient to prevent the leakage and/or escape of the radioactive particles and/or microspheres from the pouch 180 until the radiotherapy delivery device 10 is in a desired position in the target site, as described in greater detail herein. In these embodiments, the radioactive particles and/or microspheres may include iodine or yttrium isotopes, such as yttrium-90. However, other radioactive particles and/or microspheres are also contemplated and included within the scope of the present disclosure.

As further illustrated in FIG. 2A, the pouch 180 may be disposed within the lumen 138 of the catheter 130 between the distal end 132 and the proximal end 134. In some embodiments, the pouch 180 may be disposed within the lumen 138 prior to the catheter 130 being coupled to the body 100 of the radiotherapy delivery device 10. In these embodiments, the pouch 180 may be inserted into the proximal end 134 of the catheter 130 and pushed into the lumen 138 towards the distal end 132 of the catheter 130 until the pouch 180 is situated within the catheter 130. For example, the pouch 180 may be manually inserted into the proximal end 134 of the catheter 130 and a positioning mechanism, such as a pushrod or other similar mechanism, may be used to force the pouch 180 through the catheter 130 until the pouch 180 is positioned such that it may interact with the puncture shaft 140 when the puncture shaft 140 is in the advanced position. It should be understood that, in these embodiments, the pushrod may be a blunt pushrod or any similar mechanism that will not puncture the pouch 180 during positioning. Once the pouch 180 is positioned, the catheter 130 may be coupled to the body 100 of the radiotherapy delivery device 10.

In other embodiments, the catheter 130 may be coupled to the body 100 of the radiotherapy delivery device 10 prior to inserting the pouch 180 into the lumen 138 of the catheter 130. In these embodiments, the pouch 180 may be disposed in the lumen 138 by inserting the pouch 180 through the distal end 132 of the catheter 130. For example, the pouch 180 may be manually inserted into the proximal end 134 of the catheter 130 and a positioning mechanism, such as a pushrod or other similar mechanism, may be used to force the pouch 180 through the catheter 130 until the pouch 180 is positioned such that it may interact with the puncture shaft 140 when the puncture shaft 140 is in the advanced position. In other embodiments, the pouch 180 may be drawn into the catheter 130 using the syringe 120. For example, movement of the syringe 120 from the depressed position to the extended position may generate negative pressure within the catheter 130, which may act to draw the pouch into the catheter 130 (e.g., in the +x direction as shown in the coordinate axis of FIG. 2A).

In the embodiments described herein, the catheter 130 may further include at least one stop 131, which may be used to aid a user in positioning the pouch 180 within the catheter 130. In these embodiments, the at least one stop 131 may be positioned between the proximal end 134 and the distal end of the catheter 130, such that the pouch 180 engages the at least one stop 131 when the pouch 180 is inserted into the catheter 130. The at least one stop 131 may further act to secure the pouch 180 within the catheter 130 as the pouch 180 is punctured by the puncture shaft 140, as will be described in additional detail herein.

It should be noted that, although the catheter 130 is depicted as having a single stop 131, the catheter 130 may include any number of stops, such as a plurality of stops, without departing from the scope of the present disclosure. For example, the catheter 130 may include a first stop and a second stop, both of which may be positioned between the proximal end 134 and the distal end 132 of the catheter 130. In these embodiments, the pouch 180 may be inserted into the catheter 130 such the pouch 180 is positioned between the first stop and the second stop of the catheter 130.

In the embodiments described herein, the pouch 180 may further include a first surface 182 and a second surface 184. In these embodiments, the first surface 182 of the pouch 180 may correspond to the surface of the pouch 180 which is positioned towards the proximal end 134 of the catheter 130, while the second surface 184 of the pouch 180 may correspond to the surface of the pouch 180 which is positioned towards the distal end 132 of the catheter 130 when then pouch 180 has been inserted into the lumen 138 of the catheter 130. In these embodiments, the puncture shaft 140 may contact and/or pierce the first and/or second surface 182, 184 of the pouch 180 in order to release the radioactive particles and/or microspheres sealed within the pouch 180, as will be described in more detail herein.

Referring still to FIG. 2A, the radiotherapy delivery device 10 is illustrated in a first position. In this position, the pouch 180 has been inserted into the lumen 138 of the catheter 130, the puncture shaft 140 is in the retracted position, and the syringe 120 is in the extended position. As illustrated in FIG. 2A, the puncture shaft 140 may have a length LP that is generally longer than a length LB of the body 100 of the radiotherapy device 10. That is, the puncture shaft 140 extends beyond the first end 102 and the second end 104 of the body 100 of the radiotherapy delivery device 10. In addition, when assembled as described herein, the puncture shaft 140 may extend into the lumen 138 of the catheter 130 at the proximal end 134 thereof. Although the puncture shaft 140 is illustrated as having a length L_{P} which is greater than the length of the body 100, it should be understood that the puncture shaft 140 may have any length _{LP} without departing from the scope of the present disclosure.

With the radiotherapy delivery device 10 adjusted to the first position, the cutting tip 136 of the distal end 132 of the catheter 130 may be inserted into a target site, such as a tumor or diseased tissue site. In this embodiment, the distal end 132 of the catheter may further include a retractable sheath 137, which may cover the cutting tip 136 of the catheter 130 until the distal end 132 of the catheter 130 is positioned adjacent the target site. By utilizing the retractable sheath, the cutting tip 136 of the distal end 132 of the catheter 130 may be covered until the catheter 130 is appropriately positioned, which may alleviate the risk of the cutting tip 136 damaging an area of healthy tissue as the catheter 130 is advanced to the target site. In these embodiments, the radiotherapy delivery device 10 may be used in connection with an endoscope, such as a brachial endoscope, or any other similar device that may create a pathway to the target site for the radiotherapy delivery device 10. For example, the endoscope may be used to access the target site, at which point the radiotherapy delivery device may be delivered through a working lumen of the endoscope until the radiotherapy delivery device 10 is positioned adjacent the target site. Once the radiotherapy delivery device 10 is positioned adjacent the target site, the retractable sheath 137 may be withdrawn, and the cutting tip 136 may be inserted into the target site.

Turning now to FIG. 2B, the radiotherapy delivery device 10 is illustrated in a second position. In the second position, the pouch 180 remains within the lumen 138 of the catheter 130, and the syringe 120 remains in the extended position, while the puncture shaft 140 is advanced from the retracted position to the advanced position.

To translate the puncture shaft 140 from the retracted position to the advanced position, the puncture handle 142 may be moved in the longitudinal direction (−x shown in the coordinate axis of FIG. 2B) through the puncture slot 146 and towards the distal end 132 of the catheter 130. For example, a user may manually move the puncture handle 142, such as by using a thumb or index finger. Because the puncture handle 142 is connected to the puncture shaft 140, the longitudinal translation of the puncture handle 142 will similarly result in longitudinal translation of the puncture shaft 140.

As the puncture shaft 140 is advanced in the longitudinal direction towards the distal end 132 of the catheter 130, the puncture shaft 140 may advance through the lumen 138 of the catheter 130. The advance of the puncture shaft 140 may continue until the puncture shaft 140 contacts and pierces the first surface 182 and subsequently the second surface 184 of the pouch 180, as is depicted in FIG. 2B. In these embodiments, the puncture shaft 140 may include a piercing tip 148, which may be used to puncture the first and second surfaces 182, 184 of the pouch 180. It should be noted that, as the puncture shaft 140 pierces the pouch 180, the at least one stop 131 may act to ensure that the pouch 180 remains positioned within the catheter 130. Furthermore, in some embodiments, contact between the second surface 184 of the pouch 180 and the at least one stop 131 (e.g., caused by the advance of the puncture shaft 140 in the−x direction as shown in the coordinate axis of FIG. 2B) may further aid the puncture shaft 140 in piercing the pouch 180. By piercing the first and second surfaces 182, 184 of the pouch 180, the radioactive particles and/or microspheres sealed within the pouch 180 may be released from the pouch 180, such that the particles and/or microspheres may traverse the lumen 138 of the catheter 130.

Referring now to FIG. 2C, the radiotherapy delivery device 10 is depicted in a third position. In the third position, the puncture shaft 140 is returned from the advanced position to the retracted position, as indicated by the arrow displayed in FIG. 2C, and the syringe 120 is depressed to the depressed position to dispense fluid into the lumen 138 of the catheter 130. By returning the puncture shaft 140 to the retracted position prior to depressing the syringe 120, a user may ensure that the puncture shaft 140 does not interfere with the flow of fluid from the syringe 120 through the lumen 138 of the catheter 130 and/or the pouch 180. However, it should be understood that the puncture shaft 140 may also remain in the advanced position when the radiotherapy delivery device 10 is in the third position.

In the embodiment illustrated by FIG. 2C, the syringe 120 may be manually depressed from the engaged position to the depressed position, such as by using a thumb or index finger. In these embodiments, the syringe 120 may further include a plurality of markings 122 which may indicate the volume of fluid being supplied from the syringe 120 and into the lumen 138 of the catheter 130. The volume of fluid supplied to the lumen 138 may in turn correspond to the volume of radioactive particles and/or microspheres which are provided to the target site, such that a user may easily and accurately determine that a desired dosage of radioactive particles and/or microspheres are deposited at the target site. Additionally, the manual depression of the syringe 120 may further allow a user to control the rate at which fluid is provided to the lumen 138 of the catheter 130. For example, quickly and/or forcefully depressing the syringe 120 may increase the rate at which fluid is dispensed into the lumen 138 of the catheter 130. In contrast, a gradual depression of the syringe 120 may decrease the rate at which fluid is dispensed into the lumen 138 of the catheter 130. In the embodiments described herein, the fluid present in the syringe may be saline, or any other fluid suitable for use in radiotherapy procedures.

Referring still to FIG. 2C, as fluid is forced into the lumen 138 of the catheter 130 by the syringe 120, the fluid may traverse the lumen 138 towards the distal end 132 of the catheter 130. As the fluid travels towards the distal end 132 of the catheter 130, the fluid may contact the first surface 182 of the pouch 180. Because the pouch 180 has been pierced by the puncture shaft 140, the fluid may pass through the first surface 182 and the second surface 184 of the pouch 180. As has been described herein, as fluid passes through the pouch 180, the at least one stop 131 may ensure that the pouch 180 remains positioned within the catheter 130.

As the fluid traverses an interior portion of the pouch 180 between the first surface 182 and the second surface 184, the fluid may force the radioactive particles and/or microspheres contained within the pouch out of second surface 184 of the pouch 180. A user may continue to depress the syringe 120 until the flow of fluid from the syringe 120 through the lumen 138 of the catheter 130 and the pouch 180 has carried a desired volume of radioactive particles and/or microspheres out of the distal end 132 of the catheter 130 and into the target site. For example, a desired volume of radioactive particles and/or microspheres may correspond to a particular volume of particles required to perform a treatment at the target site. In these embodiments, the desired volume of radioactive particles may be a predetermined volume of radioactive particles. Accordingly, the flow of fluid from the syringe 120 to the catheter 130 may be monitored to determine when the desired volume of radioactive particles and/or microspheres has been deployed at the target site.

Referring now to FIGS. 2A-2C, the radiotherapy delivery device may be further used to provide radiotherapy to a plurality of target sites during a single radiotherapy procedure. In these embodiments, the syringe 120 may be depressed to a plurality of depressed positions, such as a first depressed position and a second depressed position, where each of the plurality of depressed positions corresponds to a different target site.

For example, the radiotherapy delivery device 10 may be moved to the first position (FIG. 2A), at which point the catheter 130 may be inserted into a first target site. With the catheter 130 positioned at the first target site, the puncture shaft 140 may be advanced towards the distal end 132 of the catheter 130 to pierce the pouch 180, such that the radioactive particles and/or microspheres sealed within the pouch 180 may be released (FIG. 2B).

Once the pouch 180 has been pierced, the syringe 120 may be moved from the extended position to a first depressed position. In these embodiments, the first depressed position may correspond with a first marker 122a indicated on the syringe 120. When the syringe 120 is depressed to the first depressed position, a volume of fluid may flow from the syringe 120 and into the lumen 138 of the catheter 130. As the fluid passes through the catheter 130, the fluid may carry radioactive particles and/or microspheres out of the pouch 180, out of the distal end 132 of the catheter 130 and into the first target site.

In some embodiments, once radioactive particles and/or microspheres have been supplied to the first target site, the catheter 130 may be repositioned to a second target site. With the catheter 130 positioned at the second target site, the syringe 120 may be further depressed from the first depressed position to a second depressed position. In these embodiments, the second depressed position may correspond with a second marker 122b indicated on the syringe 120. As the syringe 120 is depressed to the second depressed position, a volume of fluid may flow from the syringe 120 and into the lumen 138 of the catheter 130. As the fluid passes through the catheter 130, the fluid may carry additional radioactive particles and/or microspheres out of the pouch 180, out of the distal end 132 of the catheter 130 and into the second target site.

The catheter 130 may be repositioned at additional target sites, as described herein, until the radiotherapy procedure is completed, the fluid contained within the syringe 120 is exhausted, and/or the radioactive particles and/or microspheres contained within the pouch 180 are fully dispensed. In the event that each of the plurality of target sites receives the desired radiotherapy procedure, the radiotherapy delivery device 10 may be withdrawn from the target site.

In some embodiments, the fluid contained within the syringe 120 and/or the radioactive particles and/or microspheres contained within the pouch 180 may be exhausted before each of the plurality of target sites receives radiotherapy. In these embodiments, the radiotherapy delivery device 10 may require additional fluid and/or an additional pouch 180 during the radiotherapy procedure. In these instances, the syringe 120 may be removed from the body 100 of the radiotherapy delivery device 10 and refilled with fluid. Similarly, the catheter 130 may be removed from the body of the radiotherapy delivery device 10, and an additional pouch 180 may be inserted into the lumen of the catheter 130. In these embodiments, the pouch 180 which has been punctured may be removed from the catheter 130 prior to inserting the additional pouch 180. For example, a pushrod or other similar mechanism may be used to force the pouch 180 from the catheter 130 such that an additional pouch 180 may be utilized. The syringe 120 and/or pouch 180 may be refilled any number of times until each of the plurality of target sites has received treatment and the radiotherapy procedure is concluded.

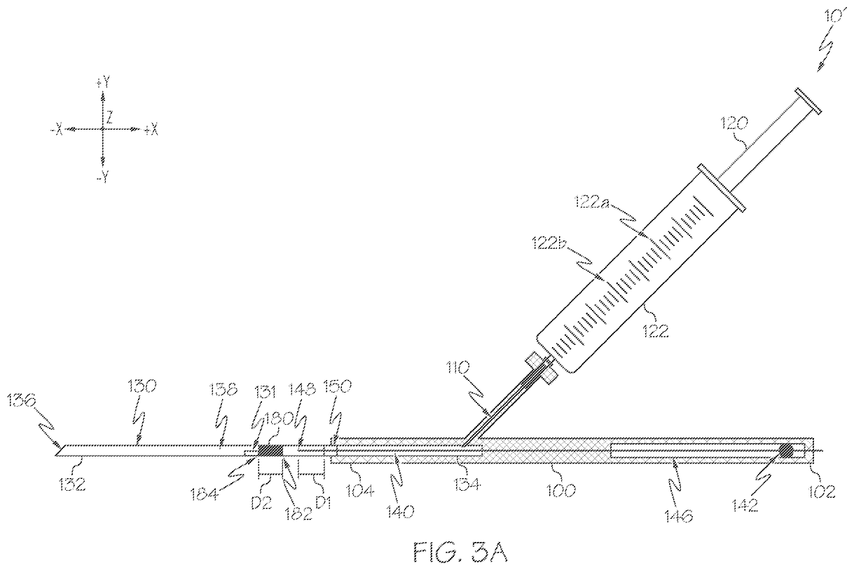

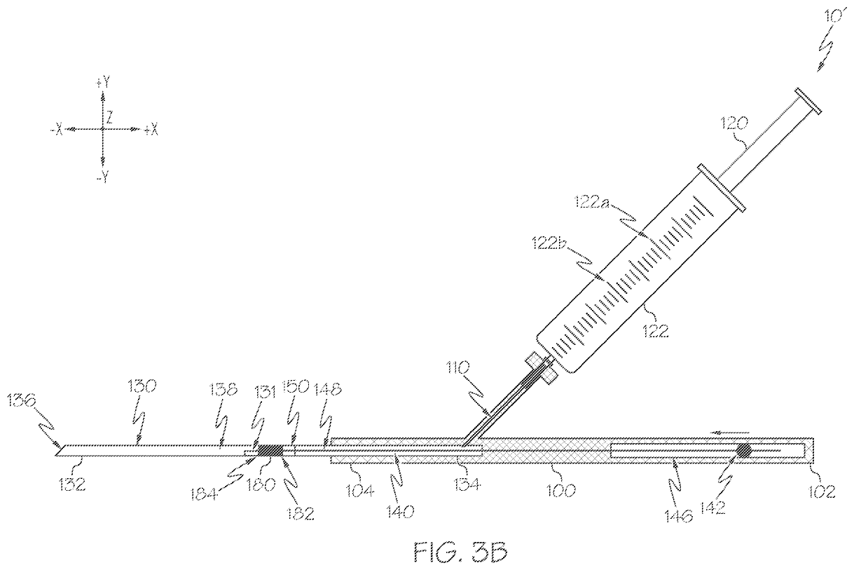

Referring now to FIG. 3A, an alternative embodiment of a radiotherapy delivery device 10′ is illustrated. It should be appreciated that the radiotherapy delivery device 10′ is similar to the radiotherapy delivery device 10 described herein. As such, like structure is indicated with like reference numerals.

In the embodiment depicted by FIG. 3A, the puncture shaft 140 of the radiotherapy delivery device 10′ may further include a piston 150, which may translate in the longitudinal (+/−x direction as shown in the coordinate axis of FIG. 3A) direction along with the puncture shaft 140. As the puncture shaft advances (e.g., moves in the-x direction towards the distal end 132 of the catheter 130) into and through the pouch 180, the piston 150 may contact the first surface 182 of the pouch 180 and force radioactive particles and/or microspheres out of the second surface 184 of the pouch 180 as the piston 150 advances.

Referring still to FIG. 3A, the radiotherapy delivery device 10′is illustrated in a first position. In this position, the pouch 180 has been inserted into the lumen 138 of the catheter 130 such that the pouch 180 contacts the at least one stop 131, the puncture shaft 140 is in the retracted position, and the syringe 120 is in the extended position. With the radiotherapy delivery device adjusted to the first position, the cutting tip 136 of the distal end 132 of the catheter 130 may be inserted into a target site.

Turning now to FIG. 3B, the radiotherapy delivery device 10′ is illustrated in a second position. In the second position, the pouch 180 remains within the lumen 138 of the catheter 130, and the syringe 120 remains in the extended position, while the puncture shaft 140 is advanced from the retracted position to the advanced position.

To translate the puncture shaft 140 from the retracted position to the advanced position, a user may manually move the puncture handle 142 in the longitudinal (−x shown in the coordinate axis of FIG. 3B) direction through the puncture slot 146 and towards the distal end 132 of the catheter 130, such as by using a thumb or index finger. Because the puncture handle 142 is connected to the puncture shaft 140, the longitudinal translation of the puncture handle 142 will similarly result in longitudinal translation of the puncture shaft 140. In these embodiments, the piston 150 may also be disposed on the puncture shaft 140, such that translation of the puncture handle 142 and puncture shaft 140 in the longitudinal direction will similarly result in translation of the piston 150 in the longitudinal direction.

In the embodiment depicted in FIGS. 3A and 3B, the piston 150 may be disposed on the puncture shaft 140 at a distance from the piercing tip 148 of the puncture shaft 140. For example, the piston 150 may be spaced from the piercing tip 148 of the puncture shaft 140 in a longitudinal direction by a first distance Di. In some embodiments, the distance Di may be equal to the length of the pouch 180, which may be defined as a second distance D2 between the first surface 182 and the second surface 184 of the pouch 180.

As the puncture shaft 140 and piston 150 are advanced in the longitudinal direction (−x direction as shown in the coordinate axis of FIG. 3B) towards the distal end 132 of the catheter 130, the puncture shaft 140 may advance through the lumen 138 of the catheter 130. The advance of the puncture shaft 140 may continue until the puncture shaft 140 contacts and pierces the first surface 182 and the second surface 184 of the pouch 180, as is depicted in FIG. 3B. As has been described herein, as the puncture shaft 140 pierces the pouch 180, the at least one stop 131 may act to ensure that the pouch 180 remains positioned within the catheter 130. Furthermore, in some embodiments, contact between the second surface 184 of the pouch 180 and the at least one stop 131 (e.g., caused by the advance of the puncture shaft 140 in the−x direction as shown in the coordinate axis of FIG. 3B) may further aid the puncture shaft 140 in piercing the pouch 180.

As further depicted in FIGS. 3A and 3B, in embodiments in which the first distance D₁ the piston 150 is spaced from the piercing tip 148 is equal to the second distance D₂ corresponding to the length of the pouch 180, it should be understood that the piercing tip 148 of the puncture shaft 140 may contact the second surface 184 of the pouch 180 at the same time the piston 150 contacts the first surface 182 of the pouch 180. As a result, the piercing tip 148 may puncture both the first and second surfaces 182, 184 of the pouch 180 prior to the piston 150 first surface 182 of the pouch.

Once the puncture shaft 140 has penetrated the pouch 180, the translation of the puncture shaft 140 in the longitudinal direction (−x direction as shown in the coordinate axis of FIG. 3B) may continue such that the piston 150 contacts the first surface 182 of the pouch 180. When the piston 150 comes into contact with the first surface 182 of the pouch 180, continued longitudinal translation of the puncture shaft 140 may cause the piston 150 to compress the first surface 182 of the pouch 180 towards the second surface 184 of the pouch 180, such that the pouch 180 is compressed between the piston 150 and the at least one stop 131. In these embodiments, the compression of the pouch 180 may force radioactive particles and/or microspheres to be deployed from the pouch 180. Continued advancement of the piston 150 in the longitudinal direction may force additional radioactive particles and/or microspheres to be deployed from the pouch 180, such that the particles and/or microspheres are pushed out of the distal end 132 of the catheter 130 and into the target site. In these embodiments, the puncture shaft 140 may include a plurality of markings which may correspond to the volume of radioactive particles and/or microspheres delivered to the target site.

Referring still to FIGS. 3A and 3B, it should be understood that the use of the piston 150 to deploy radioactive particles and/or microspheres sealed in the pouch 180 may alleviate the need for the syringe 120, such that the syringe 120 may be optional. However, the syringe 120 may still be advantageous in certain embodiments of the radiotherapy delivery device 10′. For example, the syringe 120 may be used to deploy fluid into the lumen 138 of the catheter 130 in order to clear any residual radioactive particles and/or microspheres that may become lodged in the catheter 130. Similarly, in the event the puncture shaft 140 and piston 150 have limited translation in the longitudinal direction, the syringe 120 may be used to provide fluid to the catheter 130 in order to deploy additional radioactive particles and/or microspheres that the puncture shaft 140 and piston 150 are unable to deploy.

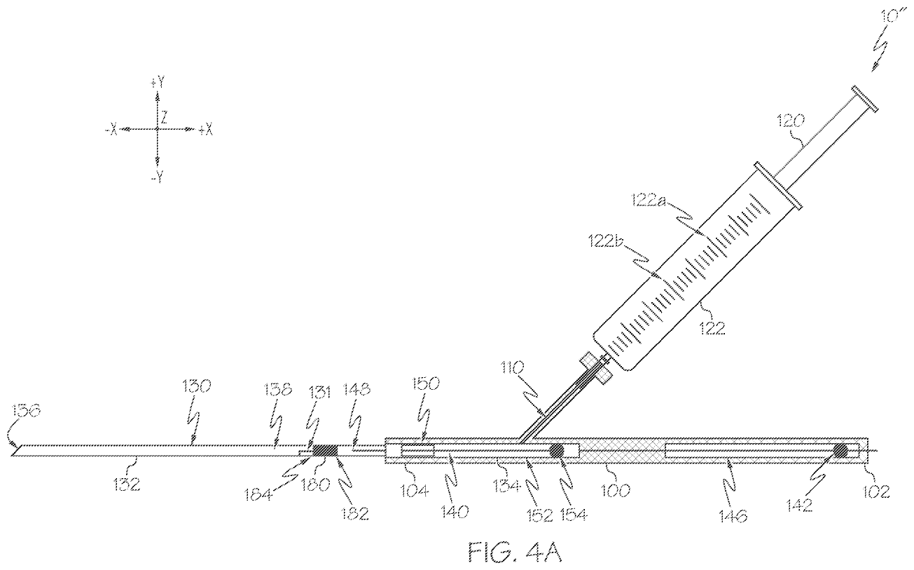

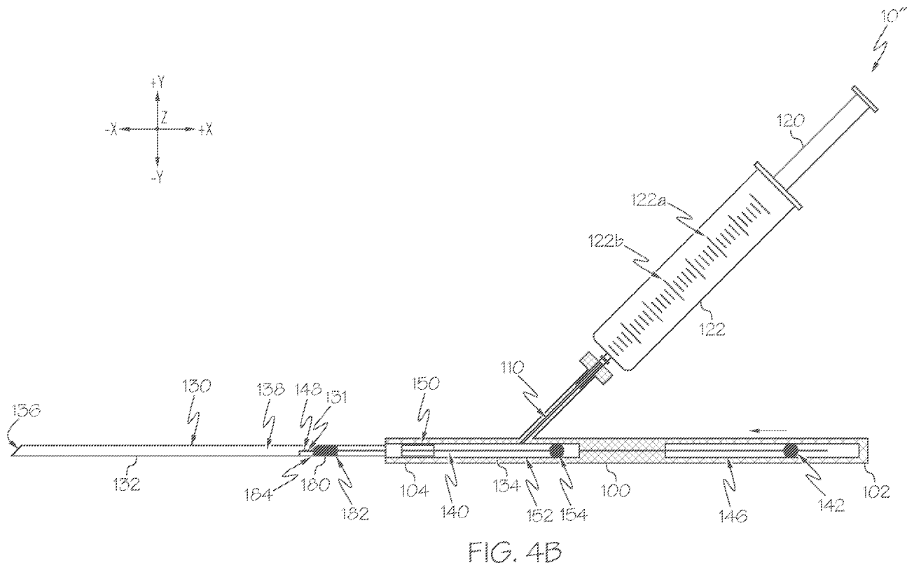

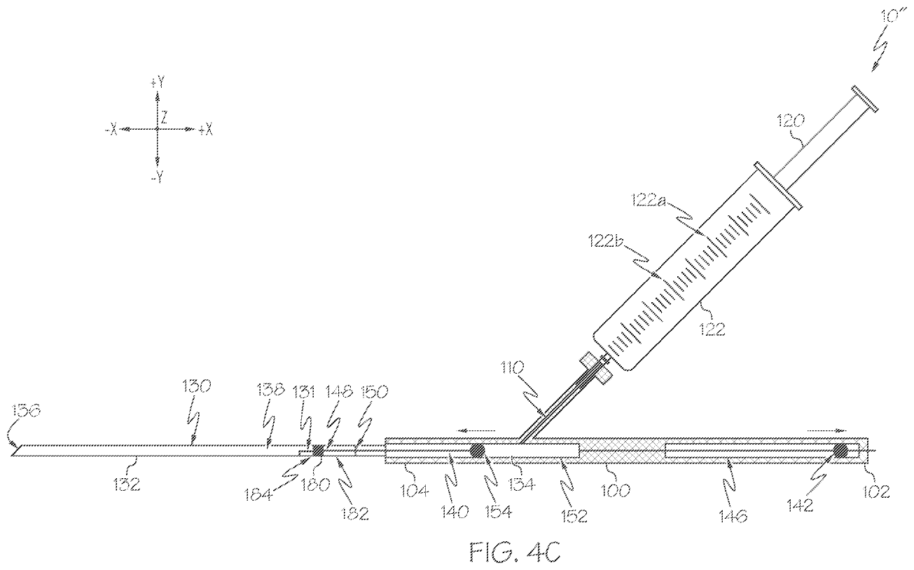

Referring now to FIG. 4A, an alternative embodiment of a radiotherapy delivery device 10″ is illustrated. It should be appreciated that the radiotherapy delivery device 10″ is similar to the radiotherapy delivery device 10 and radiotherapy delivery device 10′ described herein. As such, like structure is indicated with like reference numerals.

In the embodiment depicted by FIG. 4A, the radiotherapy delivery device 10″ may include a piston shaft 152, such that the piston 150 and the puncture shaft 140 are capable of moving independently of one another. In these embodiments, the piston 150 may be coaxially disposed about the puncture shaft 140, such that the puncture shaft 140 may move in the longitudinal direction (+/−x direction as shown in the coordinate axis of FIG. 4A) through the piston 150. Similarly, the piston 150 may be translated about the puncture shaft 140 by way of the piston shaft 152. In these embodiments, the piston 150 may be translated between an engaged position (e.g., translated towards the distal end 132 of the catheter 130 in the−x direction, as shown in the coordinate axis of FIG. 4A) and a disengaged position (e.g., translated towards the proximal end 134 of the catheter 130 in the +x direction, as shown in the coordinate axis of FIG. 4A). The piston 150 may contact the pouch 180 in the engaged position and be withdrawn from the pouch 180 in the disengaged position.

In operation, as the puncture shaft 140 advances (e.g., moves in the−x direction towards the distal end 132 of the catheter 130) into and through the piston 150, the puncture shaft 140 may contact and pierce the pouch 180. As has been noted herein, as the puncture shaft 140 pierces the pouch 180, the at least one stop 131 may act to ensure that the pouch 180 remains positioned within the catheter 130. Furthermore, in some embodiments, contact between the second surface 184 of the pouch 180 and the at least one stop 131 (e.g., caused by advancement of the puncture shaft 140 in the−x direction as shown in the coordinate axis of FIG. 4A) may further aid the puncture shaft 140 in piercing the pouch 180.

With the pouch 180 pierced, the piston 150 may be separately translated about the puncture shaft 140 by way of the piston shaft 152 until the piston 150 contacts the pouch 180 in the engaged position. The force of the piston 150 contacting the pouch 180 may cause the pouch 180 to become compressed between the piston 150 and the at least one stop 131, such that the piston 150 may force radioactive particles and/or microspheres out of the pouch 180 as the piston 150 advances. In these embodiments, the piston shaft 152 may further include a piston handle 154 for manually actuating the piston 150 in the longitudinal direction.

Referring still to FIG. 4A, the radiotherapy delivery device 10″ is illustrated in a first position. In this position, the pouch 180 has been inserted into the lumen 138 of the catheter 130, the puncture shaft 140 is in the retracted position, and the piston shaft 152 is in the disengaged position. With the radiotherapy delivery device 10″ adjusted to the first position, the cutting tip 136 of the distal end 132 of the catheter 130 may be inserted into a target site.

Turning now to FIG. 4B, the radiotherapy delivery device 10″ is illustrated in a second position. In the second position, the pouch 180 remains within the lumen 138 of the catheter 130 and the piston shaft 152 remains in the disengaged position, while the puncture shaft 140 is advanced from the retracted position to the advanced position.

To translate the puncture shaft 140 from the retracted position to the advanced position, a user may manually move the puncture handle 142 in the longitudinal (−x shown in the coordinate axis of FIG. 4B) direction through the puncture slot 146 and towards the distal end 132 of the catheter 130, such as by using a thumb or index finger. Because the puncture handle 142 is connected to the puncture shaft 140, the longitudinal translation of the puncture handle 142 will similarly result in longitudinal translation of the puncture shaft 140. As the puncture shaft 140 is advanced in the longitudinal direction (−x direction as shown in the coordinate axis of FIG. 4B) towards the distal end 132 of the catheter 130, the puncture shaft 140 may advance through the lumen 138 of the catheter 130. The advance of the puncture shaft 140 may continue until the puncture shaft 140 contacts and pierces the first surface 182 and the second surface 184 of the pouch 180, as is depicted in FIG. 4B.

Referring now to FIG. 4C, once the puncture shaft 140 has penetrated the pouch 180, the radiotherapy delivery device 10″ may be moved to a third position. In the third position, the puncture shaft 140 is returned from the advanced position to the retracted position, and the piston shaft 152 is moved from the disengaged position to the engaged position. By returning the puncture shaft 140 to the retracted position prior to engaging the piston shaft 152, a user may ensure that the puncture shaft 140 does not interfere with the flow of radioactive particles and/or microspheres from the pouch 180. However, it should be understood that the puncture shaft 140 may also remain in the advanced position when the radiotherapy delivery device 10″ is in the third position.

As described herein, the translation of the piston shaft 152 in the longitudinal direction (−x direction as shown in the coordinate axis of FIG. 4C) may be achieved by manually moving the piston handle 154 in the longitudinal (−x direction shown in the coordinate axis of FIG. 4C) direction towards the distal end 132 of the catheter 130, such as by using a thumb or index finger. Because the piston handle 154 is connected to the piston shaft 152, the longitudinal translation of the piston handle 154 will similarly result in longitudinal translation of the piston shaft 152. As the piston shaft 152 is advanced in the longitudinal direction (−x direction as shown in the coordinate axis of FIG. 4B) towards the distal end 132 of the catheter 130, the piston shaft 152 may advance through the lumen 138 of the catheter 130.

The advance of the piston shaft 152 may continue such that the piston 150 contacts the first surface 182 of the pouch 180, at which point the piston shaft 152 may be considered to be in the engaged position. When the piston 150 comes into contact with the first surface 182 of the pouch 180, continued longitudinal translation of the piston shaft 152 may cause the piston 150 to compress the first surface 182 of the pouch 180 towards the second surface 184 of the pouch 180, such that the pouch 180 becomes compressed between the piston 150 and the at least one stop 131. In these embodiments, the compression of the pouch 180 may force radioactive particles and/or microspheres to be deployed from the pouch 180. Continued advancement of the piston 150 in the longitudinal direction may force additional radioactive particles and/or microspheres to be deployed from the pouch 180, such that the particles and/or microspheres are pushed out of the distal end 132 of the catheter 130 and into the target site. In these embodiments, the piston shaft 152 may include a plurality of markings which may correspond to the volume of radioactive particles and/or microspheres delivered to the target site. It should be noted that the volume of radioactive particles and/or microspheres delivered to the target site may be more easily controlled in the embodiments depicted in FIGS. 4A-4C, in which the piston 150 and puncture shaft 140 may move independently from one another.

Referring still to FIGS. 4A-4C, it should be understood that the use of the piston 150 to deploy radioactive particles and/or microspheres sealed in the pouch 180 may alleviate the need for the syringe 120, such that the syringe 120 may be optional. However, the syringe 120 may still be advantageous in certain embodiments of the radiotherapy delivery device 10″. As previously described, the syringe 120 may be used to deploy fluid into the lumen 138 of the catheter 130 in order to clear any residual radioactive particles and/or microspheres that may become lodged in the catheter 130. Similarly, in the event the puncture shaft 140 and piston 150 have limited translation in the longitudinal direction, the syringe 120 may be used to provide fluid to the catheter 130 in order to deploy additional radioactive particles and/or microspheres that the puncture shaft 140 and piston 150 are unable to deploy.

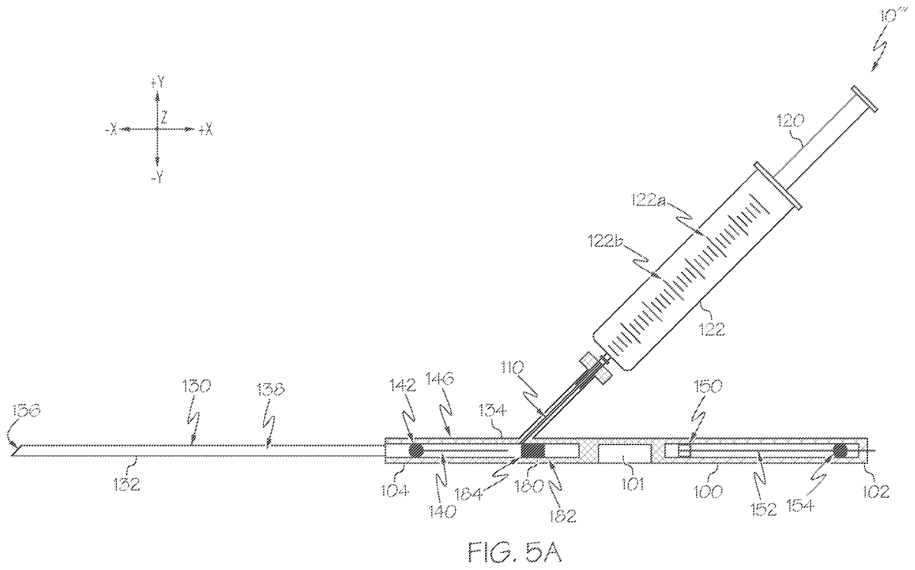

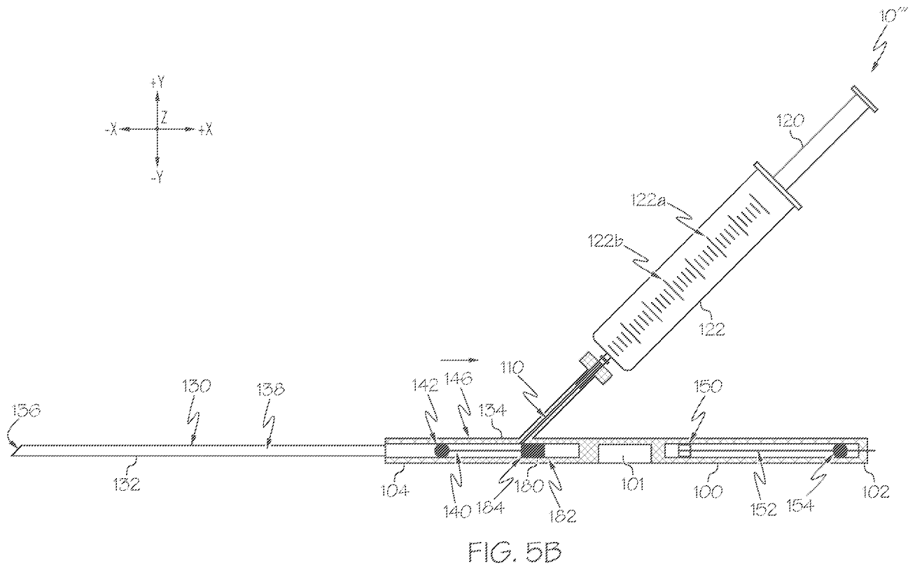

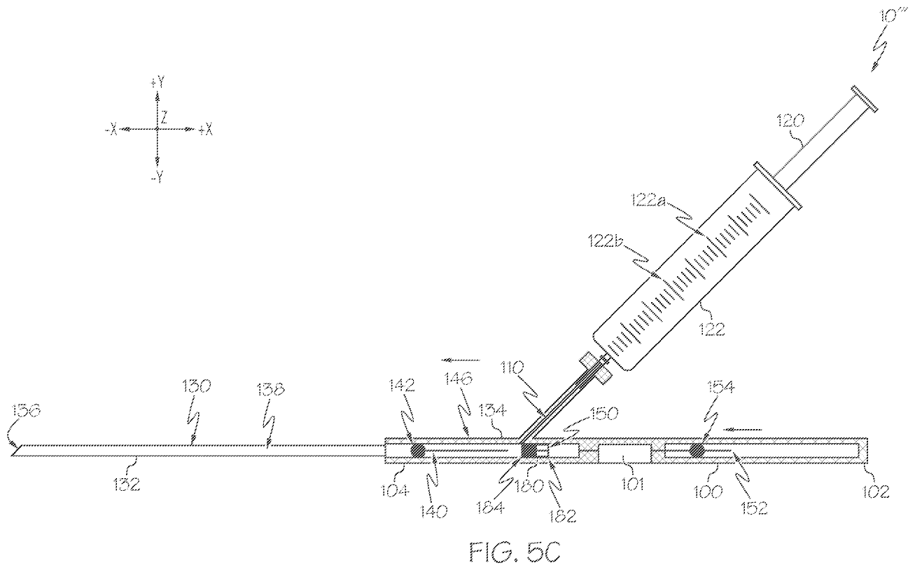

Referring now to FIG. 5A, an alternative embodiment of a radiotherapy delivery device 10′″ is illustrated. It should be appreciated that the radiotherapy delivery device 10′″ is similar to the previous radiotherapy devices described herein. As such, like structure is indicated with like reference numerals.

In the embodiment depicted by FIG. 5A, the radiotherapy delivery device 10′″ may include a puncture shaft 140 positioned in a second end 104 of a body 100 of the radiotherapy delivery device 10′″ and a piston shaft 152 positioned in a first end 102 of the body 100 of the radiotherapy delivery device 10′″, such that the piston shaft 152 and the puncture shaft 140 are capable of moving independently of one another.

In these embodiments, the piston shaft 152 may further include a piston 150 coaxially disposed about the piston shaft 152, such that the piston 150 may move in the longitudinal direction (+/−x direction as shown in the coordinate axis of FIG. 5A) through the body 100. In these embodiments, the piston 150 may be translated between an engaged position (e.g., translated towards the distal end 132 of the catheter 130 in the−x direction, as shown in the coordinate axis of FIG. 5A) and a disengaged position (e.g., translated towards the proximal end 134 of the catheter 130 in the +x direction, as shown in the coordinate axis of FIG. 5A).

Referring still to FIG. 5A, a pouch 180 may be positioned between the puncture shaft 140 and the piston shaft 152, such that the puncture shaft 140 and the piston shaft 152 move in opposite longitudinal directions to engage the pouch 180. For example, the puncture shaft 140 may move towards the first end 102 of the body 100 (e.g., in the +x direction as depicted in the coordinate axis of FIG. 5A) to engage a second surface 184 of the pouch 180, while the piston shaft 152 may move towards the second end 104 of the body 100 (e.g., in the−x direction as depicted in the coordinate axis of FIG. 5A) to engage a first surface 182 of the pouch 180.

Referring still to FIG. 5A, in order to position the pouch 180 between the puncture shaft 140 and the piston shaft 152, the first end 102 and the second end 104 of the body 100 may be formed as separate components that may be releasbly coupled. In these embodiments, the pouch 180 may be positioned within either the first end 102 of the second end 104 of the body 100 prior to the first end 102 and second end 104 of the body being coupled together. The first end 102 and the second end 104 may be threadably coupled, or may utilize any other similar releasable coupling.

In other embodiments, the body 100 may be formed as a single, monolithic structure. In these embodiments, the body 100 may further include a chamber 101 disposed between the first end 102 of the body and the second end 104 of the body 100 that may be used for accessing an interior portion of the body 100, as is depicted in FIG. 5A. In these embodiments, the chamber 101 may have a releasable cover that may be moved between an open position and a closed position. When the cover is in the open position, a user may load the pouch 180 into the interior of the body 100, and may secure the pouch 180 in the body by moving the cover to the closed position.

In operation, as the puncture shaft 140 advances (e.g., moves in the−x direction towards the distal end 132 of the catheter 130) the puncture shaft 140 may contact and pierce the second surface 184 of the pouch 180. With the pouch 180 pierced, the piston 150 may be separately translated by way of the piston shaft 152 until the piston 150 contacts the first surface 182 of the pouch 180 in the engaged position. The force of the piston 150 contacting the first surface 182 of pouch 180 may force radioactive particles and/or microspheres out of the second surface 184 of the pouch 180 as the piston 150 advances. In these embodiments, the piston shaft 152 may further include a piston handle 154 for manually actuating the piston 150 in the longitudinal direction.

Referring still to FIG. 5A, the radiotherapy delivery device 10′″ is illustrated in a first position. In this position, the pouch 180 has been inserted into the body 100 of the radiotherapy delivery device 10″, the puncture shaft 140 is in the retracted position, and the piston shaft 152 is in the disengaged position. With the radiotherapy delivery device 10′″ adjusted to the first position, the cutting tip 136 of the distal end 132 of the catheter 130 may be inserted into a target site.

Turning now to FIG. 5B, the radiotherapy delivery device 10′″ is illustrated in a second position. In the second position, the pouch 180 remains within the body 100 of the radiotherapy delivery device 10′″ and the piston shaft 152 remains in the disengaged position, while the puncture shaft 140 is advanced from the retracted position to the advanced position.

To translate the puncture shaft 140 from the retracted position to the advanced position, a user may manually move the puncture handle 142 in the longitudinal (+x shown in the coordinate axis of FIG. 5B) direction through the puncture slot 146 and towards the first end 102 of the body 100, such as by using a thumb or index finger. Because the puncture handle 142 is connected to the puncture shaft 140, the longitudinal translation of the puncture handle 142 will similarly result in longitudinal translation of the puncture shaft 140. As the puncture shaft 140 is advanced in the longitudinal direction (+x direction as shown in the coordinate axis of FIG. 5B) towards the first end 102 of the body 100, the puncture shaft 140 may advance until the puncture shaft 140 contacts and pierces the second surface 184 of the pouch 180, as is depicted in FIG. 5B.

Referring now to FIG. 5C, once the puncture shaft 140 has penetrated the second surface 184 of the pouch 180, the radiotherapy delivery device 10′″ may be moved to a third position. In the third position, the puncture shaft 140 is returned from the advanced position to the retracted position, and the piston shaft 152 is moved from the disengaged position to the engaged position. By returning the puncture shaft 140 to the retracted position prior to engaging the piston shaft 152, a user may ensure that the puncture shaft 140 does not interfere with the flow of radioactive particles and/or microspheres from the pouch 180. However, it should be understood that the puncture shaft 140 may also remain in the advanced position when the radiotherapy delivery device 10′″ is in the third position.

As described herein, the translation of the piston shaft 152 in the longitudinal direction (−x direction as shown in the coordinate axis of FIG. 5C) may be achieved by manually moving the piston handle 154 in the longitudinal (−x shown in the coordinate axis of FIG. 4C) direction towards the second end 104 of the body 100, such as by using a thumb or index finger. Because the piston handle 154 is connected to the piston shaft 152, the longitudinal translation of the piston handle 154 will similarly result in longitudinal translation of the piston shaft 152. As the piston shaft 152 is advanced in the longitudinal direction (−x direction as shown in the coordinate axis of FIG. 4B) towards the second end 104 of the body 100, the piston shaft 152 may advance towards the pouch 180.

The advance of the piston shaft 152 may continue such that the piston 150 contacts the first surface 182 of the pouch 180, at which point the piston shaft 152 may be considered to be in the engaged position. When the piston 150 comes into contact with the first surface 182 of the pouch 180, continued longitudinal translation of the piston shaft 152 may cause the piston 150 to compress the first surface 182 of the pouch 180 towards the second surface 184 of the pouch 180. In these embodiments, the compression of the first surface 182 of the pouch 180 may force radioactive particles and/or microspheres to be deployed from the second surface 184 of the pouch 180. Continued advancement of the piston 150 in the longitudinal direction may force additional radioactive particles and/or microspheres to be deployed from the pouch 180, such that the particles and/or microspheres are pushed out of the distal end 132 of the catheter 130 and into the target site. In these embodiments, the piston shaft 152 may include a plurality of markings which may correspond to the volume of radioactive particles and/or microspheres delivered to the target site. It should be noted that the volume of radioactive particles and/or microspheres delivered to the target site may be more easily controlled in the embodiments depicted in FIGS. 5A-5C, in which the piston 150 and puncture shaft 140 may move independently from one another.

Referring still to FIGS. 5A-5C, it should be understood that the use of the piston 150 to deploy radioactive particles and/or microspheres sealed in the pouch 180 may alleviate the need for the syringe 120, such that the syringe 120 may be optional. However, the syringe 120 may still be advantageous in certain embodiments of the radiotherapy delivery device 10′″. As previously described, the syringe 120 may be used to deploy fluid into the lumen 138 of the catheter 130 in order to clear any residual radioactive particles and/or microspheres that may become lodged in the catheter 130. Similarly, in the event the puncture shaft 140 and piston 150 have limited translation in the longitudinal direction, the syringe 120 may be used to provide fluid to the catheter 130 in order to deploy additional radioactive particles and/or microspheres that the puncture shaft 140 and piston 150 are unable to deploy.

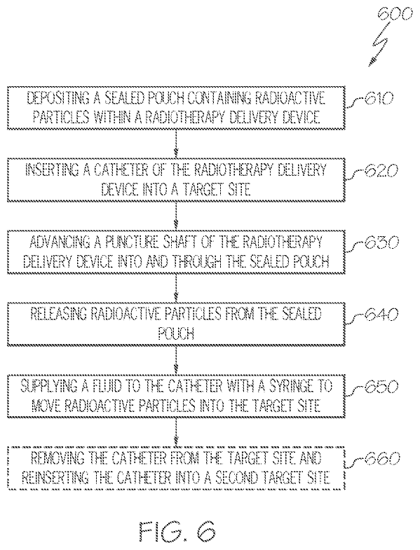

Turning now to FIG. 6, a flow chart of an illustrative method 600 of performing a radiotherapy procedure using the various devices described herein is depicted. The method 600 may initially involve depositing a sealed pouch containing radioactive particle within a radiotherapy delivery device, as is depicted at block 610. In these embodiments, the radiotherapy delivery device may include a body having a first end and a second end and a catheter having a lumen extending between a proximal end and a distal end, with the proximal end of the catheter being coupled to the second end of the body. The catheter may further include at least one stop, which may be used to aid a user in positioning the sealed pouch within the catheter. In these embodiments, the at least one stop may be positioned between the proximal end and the distal end of the catheter, such that the pouch engages the at least one stop when a user inserts the pouch into the catheter.

With the sealed pouch of radioactive particles being deposited within the radiotherapy delivery device, the method may move to block 620, which may involve inserting the catheter into a target area. In these embodiments, the catheter may include a tip, such as a cutting tip, that may be used to puncture the target area such that the catheter may be positioned in a desired location.

Once the catheter has been appropriately positioned, the method may move to block 630, which may include advancing a puncture shaft coupled to the first end of the body into and through the sealed pouch. In these embodiments, as the puncture shaft moves through the sealed pouch, the puncture shaft may form an opening, or hole, in at least one side of the sealed pouch, such that the radioactive particles contained within the sealed pouch are able to be dispersed.

At this point, the radioactive particles may be released from the sealed pouch, as is depicted at block 640. The method may then move to block 650, which may involve supplying a fluid to the catheter lumen with a syringe such that the radioactive particles are moved through the catheter lumen and into the target site. In these embodiments, the fluid supplied to the catheter lumen from the syringe may determine the volume of radioactive particles supplied to the target site, as has been described in detail herein.

In some embodiments, the method 600 may optionally include the step of removing the catheter from the target site and reinserting the catheter into a second target site, as is depicted at block 660. In these embodiments, the radiotherapy delivery device may be used to treat multiple areas in a single procedure. Furthermore, it should be noted that the catheter may be removed from the target site and reinserted into the second target area without removing the catheter from the subject, which may alleviate common issues, such as pneumothorax, that may arise from completely removing the catheter from the subject.

As should be understood in view of the foregoing, a radiotherapy delivery device and methods of performing radiotherapy are described herein. The radiotherapy delivery device may include a body having a first end a second end, and a port for receiving a syringe. The radiotherapy delivery device may further include a catheter, which may be coupled to the second end of the body. A puncture shaft may be coaxially disposed within the body, and may include a puncture handle which may be slidably coupled to a slot formed on an outer surface of the body. The puncture shaft may be translated in a longitudinal direction through the slot, which may in turn advance the puncture shaft through the body of the catheter when the catheter is coupled to the second end of the body. The syringe may include a syringe needle, which may extend through the port and into the lumen of the catheter, such that the syringe and lumen are fluidly coupled. A pouch containing radioactive particles and/or microspheres may be enclosed within a lumen of the catheter. As the puncture shaft is advanced through the body and catheter, the puncture shaft may contact the pouch, such that the puncture shaft pierces the pouch and releases the radioactive particles and/or microspheres sealed within the pouch. The syringe may be depressed to transfer fluid from the syringe and into the lumen of the catheter. As the fluid flows through the catheter, the advancing fluid may force a volume of radioactive particles and/or microspheres out of the catheter and into a target site. Embodiments of the present disclosure may be specifically advantageous for providing radiotherapy to a plurality of target sites during a single radiotherapy procedure. By controlling the dosage of radioactive particles and/or microspheres at a target site using fluid from a syringe, a user may easily and accurately deploy a desired volume of radioactive particles and/or microspheres at a particular target site.

Embodiments may be further described with references to the following numbered clauses:
Clause 1. A radiotherapy delivery device comprising: a body having a first end and a second end; a catheter having a lumen extending between a proximal end and a distal end, the proximal end being coupled to the second end of the body and the distal end including a cutting tip configured to penetrate a target site; a sealed pouch containing radioactive particles positioned within the lumen of the catheter; a puncture shaft positioned coaxially within the body, the puncture shaft configured to advance in a longitudinal direction from the first end of the body towards the second end of the body such that the puncture shaft advances into and through the sealed pouch and releases the radioactive particles; and a syringe fluidly coupled to the lumen of the catheter, such that the radioactive particles released by the sealed pouch are deposited into the target site when fluid is supplied from the syringe and into the lumen of the catheter.
Clause 2. The radiotherapy delivery device of claim 1, wherein the body further comprises a puncture slot for limiting the advance of the puncture shaft in the longitudinal direction.
Clause 3. The radiotherapy delivery device of claim 2, wherein the puncture shaft further includes a puncture handle for manually translating the puncture shaft in the longitudinal direction.
Clause 4. The radiotherapy delivery device of claim 1, wherein the body further comprises a port for fluidly coupling the syringe to the catheter body.
Clause 5. The radiotherapy delivery device of claim 1, wherein the puncture shaft extends beyond the first end and the second end of the body.
Clause 6. The radiotherapy delivery device of claim 1, wherein the sealed pouch includes iodine or yttrium isotopes.
Clause 7. The radiotherapy delivery device of claim 1, wherein the syringe is movable between an extended position and a depressed position and the syringe supplies fluid to the lumen of the catheter in the depressed position.
Clause 8. The radiotherapy delivery device of claim 7, wherein the syringe further comprises a plurality of markings that indicate a volume of fluid being supplied to the lumen of the catheter in the depressed position.
Clause 9. The radiotherapy delivery device of claim 1, wherein the catheter further comprises a retractable sheath that covers the cutting tip when the catheter is inserted into the target site.
Clause 10. The radiotherapy delivery device of claim 1, wherein the puncture shaft further comprises a piercing tip that pierces the sealed pouch.
Clause 11. The radiotherapy delivery device of claim 1, wherein the puncture shaft further comprises a piston fixed to the puncture shaft, such that the piston is translated in the longitudinal direction simultaneously with the puncture shaft.
Clause 12. The radiotherapy delivery device of claim 11, wherein the piston is positioned a first distance from the piercing tip.
Clause 13. The radiotherapy delivery device of claim 1, further comprising a piston shaft coaxially positioned within the body, the piston shaft including a piston and being configured to translate between an engaged position and a disengaged position.
Clause 14. The radiotherapy delivery device of claim 13, wherein the piston shaft further includes a piston handle for manually translating the piston shaft between the engaged position and the disengaged position.
Clause 15. The radiotherapy delivery device of claim 14, wherein the piston contacts the sealed pouch in the engaged position, such that the longitudinal movement of the piston towards the pouch releases radioactive particles from the pouch.
Clause 16. A radiotherapy delivery device comprising: a body having a first end and a second end; a catheter having a lumen extending between a proximal end and a distal end, the proximal end being coupled to the second end of the body and the distal end including a cutting tip configured to penetrate a target site; a sealed pouch containing radioactive particles positioned within the lumen of the catheter; a piston shaft positioned coaxially within the body, the piston shaft having a piston and configured to translate in a longitudinal direction between an engaged position in which the piston contacts the sealed pouch, and a disengaged position in which the piston is withdrawn from the sealed pouch; a puncture shaft positioned coaxially within the piston shaft, the puncture shaft configured to advance in the longitudinal direction from the first end of the body towards the second end of the body such that the puncture shaft advances into and through the sealed pouch and releases the radioactive particles; wherein, in the engaged position, advancement of the piston shaft in the longitudinal direction pushes radioactive particles from the sealed pouch and towards the target site.
Clause 17. The radiotherapy delivery device of claim 16, wherein the body further comprises a puncture slot for limiting the advance of the puncture shaft in the longitudinal direction.
Clause 18. The radiotherapy delivery device of claim 16, wherein the body further comprises a piston slot for limiting the translation of the piston shaft in the longitudinal direction.
Clause 19. The radiotherapy delivery device of claim 16, wherein the puncture shaft further includes a puncture handle for manually translating the puncture shaft in the longitudinal direction and the piston shaft further includes a piston handle for manually translating the piston shaft in the longitudinal direction.
Clause 20. A method of radiotherapy delivery comprising: depositing a sealed pouch containing radioactive particles within a radiotherapy delivery device including a body having a first end and a second end and a catheter having a lumen extending between a proximal end and a distal end, wherein the proximal end of the catheter is coupled to the second end of the body; inserting the catheter into a target site; advancing a puncture shaft coupled to the first end of the body into and through the sealed pouch; releasing the radioactive particles in the sealed pouch; and supplying a fluid to the catheter lumen with a syringe such that the radioactive particles are moved through the catheter lumen and into the target site.
Clause 21. The method of claim 20, wherein the puncture shaft comprises a piston for moving the radioactive particles through the catheter lumen and into the target site.
Clause 22. The method of claim 21, wherein the puncture shaft and piston are configured to move independently of one another.
Clause 23. The method of claim 20, further comprising removing the catheter from the target site and reinserting the catheter into a second target site.

As should be appreciated in view of the foregoing, a radiotherapy delivery device is disclosed. The radiotherapy delivery device may include a body having a first end a second end, and a port for receiving a syringe. The radiotherapy delivery device may further include a catheter, which may be coupled to the second end of the body. A puncture shaft may be coaxially disposed within the body, and may include a puncture handle which may be slidably coupled to a slot formed on an outer surface of the body. The puncture shaft may be translated in a longitudinal direction through the slot, which may in turn advance the puncture shaft through the body and the catheter when the catheter is coupled to the second end of the body. The syringe may include a syringe needle, which may extend through the port and into the lumen of the catheter, such that the syringe and lumen are fluidly coupled. The radiotherapy device may be specifically advantageous for providing radiotherapy to multiple target sites during a single radiotherapy procedure.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A radiotherapy delivery device, comprising:
a body having a first end and a second end;
a catheter having a lumen extending between a proximal end and a distal end, the proximal end being coupled to the second end of the body and the distal end including a cutting tip configured to penetrate a target site;
a sealed pouch containing radioactive particles positioned within the lumen of the catheter;
a puncture shaft positioned coaxially within the body, the puncture shaft configured to advance in a longitudinal direction from the first end of the body towards the second end of the body such that the puncture shaft advances into and through the sealed pouch and releases the radioactive particles; and
a syringe fluidly coupled to the lumen of the catheter, such that the radioactive particles released by the sealed pouch are deposited into the target site when fluid is supplied from the syringe and into the lumen of the catheter.

2. The radiotherapy delivery device of claim 1, wherein the body further comprises a puncture slot for limiting the advance of the puncture shaft in the longitudinal direction.

3. The radiotherapy delivery device of claim 2, wherein the puncture shaft further includes a puncture handle for manually translating the puncture shaft in the longitudinal direction.

4. The radiotherapy delivery device of claim 1, wherein the body further comprises a port for fluidly coupling the syringe to the catheter.

5. The radiotherapy delivery device of claim 1, wherein the puncture shaft extends beyond the first end and the second end of the body.

6. The radiotherapy delivery device of claim 1, wherein the sealed pouch includes iodine or yttrium isotopes.

7. The radiotherapy delivery device of claim 1, wherein the syringe is movable between an extended position and a depressed position and the syringe supplies fluid to the lumen of the catheter in the depressed position.

8. The radiotherapy delivery device of claim 7, wherein the syringe further comprises a plurality of markings that indicate a volume of fluid being supplied to the lumen of the catheter in the depressed position.

9. The radiotherapy delivery device of claim 1, wherein the catheter further comprises a retractable sheath that covers the cutting tip when the catheter is inserted into the target site.

10. The radiotherapy delivery device of claim 1, wherein the puncture shaft further comprises a piercing tip that pierces the sealed pouch.

11. The radiotherapy delivery device of claim 10, wherein the puncture shaft further comprises a piston fixed to the puncture shaft, such that the piston is translated in the longitudinal direction simultaneously with the puncture shaft.

12. The radiotherapy delivery device of claim 11, wherein the piston is positioned a first distance from the piercing tip.

13. The radiotherapy delivery device of claim 1, further comprising a piston shaft coaxially positioned within the body, the piston shaft including a piston and being configured to translate between an engaged position and a disengaged position.

14. The radiotherapy delivery device of claim 13, wherein the piston shaft further includes a piston handle for manually translating the piston shaft between the engaged position and the disengaged position.

15. The radiotherapy delivery device of claim 14, wherein the piston contacts the sealed pouch in the engaged position, such that movement of the piston in the longitudinal direction towards the sealed pouch releases the radioactive particles from the sealed pouch.

16. A radiotherapy delivery device, comprising:
a body having a first end and a second end;
a catheter having a lumen extending between a proximal end and a distal end, the proximal end being coupled to the second end of the body and the distal end including a cutting tip configured to penetrate a target site;
a sealed pouch containing radioactive particles positioned within the lumen of the catheter;
a piston shaft positioned coaxially within the body, the piston shaft having a piston and configured to translate in a longitudinal direction between an engaged position in which the piston contacts the sealed pouch, and a disengaged position in which the piston is withdrawn from the sealed pouch; and
a puncture shaft positioned coaxially within the piston shaft, the puncture shaft configured to advance in the longitudinal direction from the first end of the body towards the second end of the body such that the puncture shaft advances into and through the sealed pouch and releases the radioactive particles,
wherein, in the engaged position, advancement of the piston shaft in the longitudinal direction pushes the radioactive particles from the sealed pouch and towards the target site.

17. The radiotherapy delivery device of claim 16, wherein the body further comprises a puncture slot for limiting the advance of the puncture shaft in the longitudinal direction.

18. The radiotherapy delivery device of claim 16, wherein the body further comprises a piston slot for limiting translation of the piston shaft in the longitudinal direction.

19. The radiotherapy delivery device of claim 16, wherein the puncture shaft further includes a puncture handle for manually translating the puncture shaft in the longitudinal direction and the piston shaft further includes a piston handle for manually translating the piston shaft in the longitudinal direction.

20. (canceled)

21. (canceled)

22. (canceled)

23. (canceled)
